(19) United States
(12) Patent Application Publication
(10) Pub. No.: US 2005/0002976 A1
(43) Pub. Date: Jan. 6, 2005

(54) Polyol-in-silicone emulsions

(75) Inventor: Jianxin Wu, Higashinada-ku (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/866,942

(22) Filed: Jun. 14, 2004

Related U.S. Application Data
(60) Provisional application No. 60/479,967, filed on Jun. 19, 2003.

(51) Int. Cl. A61K 7/11

(52) U.S. Cl. 424/401

(57) ABSTRACT

Disclosed is a polyol-in-silicone emulsion comprising: (a) a continuous silicone phase; (b) a discontinuous polyol phase; (c) a branched polyether-polydiorganosiloxane emulsifier; (d) an alkyl dimethicone copolymer emulsifier; and (e) a thickener for stabilizing the composition.

### CROSS REFERENCE TO RELATED APPLICATION

This application claims the benefit of U.S. Provisional Application No. 60/479,967, filed on Jun. 19, 2003.

### FIELD OF THE INVENTION

The present invention relates to polyol-in-silicone emulsions having improved stability and topical compositions comprising thereof. The present invention further relates to such emulsions and compositions which are electrostatically-sprayable on the skin, and to methods of treating the skin by electrostatic application of such compositions.

### BACKGROUND OF THE INVENTION

The use of polyol-in-silicone emulsions in the cosmetic and skin care field has become increasingly popular due to their ability in wear resistance and fresh feel on the skin. A polyol-in-silicone emulsion comprises a polyhydric alcohol as the discontinuous phase, and a silicone component as the continuous phase. For emulsifying the emulsion, a silicone-containing emulsifier is typically used. Japanese Patent publications A-2001-39819, A-2002-179548, A-2002-179797 and A-2003-81758, and PCT publication WO 02/55588 suggest the use of branched polyether modified silicones for providing emulsions.

Elements that denote stability of an emulsion are, for example, smaller emulsion droplet size, stability at different temperature, stability over a longer period of time, lack of separation, and consistent viscosity. For providing polyol-in-silicone emulsions that are stable even when encompassing additional components such as: powders for providing color or skin feel benefit, film forming polymers for wear resistance, or skin active agents; an emulsion have improved stability over a variety of formulations is desired.

Electrostatic spraying of color cosmetics and skin care compositions has been proposed as a means for more efficient consumption and product activity, control over application, ease and cleanliness/hygiene of application, and improved finish. Electrostatic sprayable compositions are disclosed in PCT publications WO 01/12137, WO 01/12138, WO 01/12139, and WO 01/12152, and electrostatic spray devices suitable for spraying such compositions are disclosed in PCT publications WO02/55210, WO02/55211, WO02/55212, and WO02/55209.

Stability of emulsions for electrostatic sprayable compositions is particularly important, in that electrical charging of the fluid may enhance separation. Stability of compositions comprising emulsions and additional powders are particularly challenging, as such compositions tend to separate, and the powder may settle out. The resultant non-homogeneous fluid exhibits poor spray quality when sprayed electrostatically. An emulsion having improved stability such that can be electrostatically sprayed is also desired.

Based on the foregoing, there is a need for a polyol-in-silicone emulsion which has improved stability and which can be incorporated into a wide range of compositions. There is further a need for color cosmetic and skin care compositions which can be electrostatically sprayed.

### SUMMARY OF THE INVENTION

The present invention is directed to a polyol-in-silicone emulsion composition comprising:
a) a continuous silicone phase
b) a discontinuous polyol phase
c) a branched polyether-polydiorganosiloxane emulsifier;
d) an alkyl dimethicone copolymer emulsifier; and
e) a thickener for stabilizing the composition.

In another aspect, the present invention is directed to color cosmetic and skin care compositions comprising the aforementioned emulsion and other components such as powder, film forming agent, or skin active agent.

In yet another aspect, the present invention is directed to the aforementioned emulsion wherein the continuous silicone phase is a liquid insulating material; the discontinuous polyol phase is a conductive material; and wherein the emulsion is electrostatically sprayable.

These and other features, aspects, and advantages of the present invention will become evident to those skilled in the art from a reading of the present disclosure with the appended claims.

### DETAILED DESCRIPTION

While the specification concludes with claims particularly pointing out and distinctly claiming the invention, it is believed that the present invention will be better understood from the following description.

All cited references are incorporated herein by reference in their entireties. Citation of any reference is not an admission regarding any determination as to its availability as prior art to the claimed invention.

Herein, “comprising” means that other elements which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

Silicone Phase

The present emulsion comprises a continuous silicone phase in an amount sufficient to provide a stable polyol-in-silicone emulsion. Preferably, the continuous silicone phase is comprised in a total amount of from about 2% to about 90%, more preferably about 5% to about 85%, still preferably about 10% to about 80%, of the polyol-in-silicone emulsion. The silicone phase is a liquid that may be volatile or non-volatile based on the desired characteristic of the product in which the emulsion is comprised. As used herein, “volatile” means that the material has a measurable vapor pressure at 1 atm.

Suitable volatile silicones include cyclic polyalkylsiloxanes represented by the chemical formula [SiR₂—O]ₙ wherein R is an alkyl group (preferably R is methyl or ethyl, more preferably methyl) and n is an integer from about 3 to about 8, more preferably n is an integer from about 3 to about 7, and most preferably n is an integer from about 4 to about 6. When R is methyl, these materials are typically referred to as cyclomethicones. Commercially available cyclomethicones include DC244, DC344, DC245, DC345 from Dow Corning Corporation, KF994, KF995, KF996, KF9956 from Shin-Etsu Chemical, TSF404, TSF405, TSF406 from GE-Toshiba Silicone. DC245, KF995 and TSF405 are preferred cyclomethicones.

Other suitable volatile silicones are linear polydimethyl siloxanes having from about 3 to about 9 silicon atoms and the general formula (CH₃)₃ Si—O—[—Si (CH₃)₂—O—]—ₙ—Si (CH₃)₃ where n=0-7. These silicones are available from various sources including Dow Corning Corporation, Shin-Etsu Chemical and General Electric.

Polyol Phase

The present emulsion comprises a discontinuous polyol phase in an amount which can be emulsified in a stable manner in the polyol-in-silicone emulsion. Preferably, the discontinuous silicone phase is comprised in a total amount of from about 2% to about 90%, more preferably about 5% to about 80%, still preferably about 7% to about 70%, of the polyol-in-silicone emulsion. The polyol phase comprises predominantly polyhydric alcohols, and may also comprise other polar solvents which are completely soluble with the polyhydric alcohol. The components of the polyol phase may be volatile or non-volatile.

Suitable for the polyol phase are polyols such as propylene glycol, butylene glycol, dipropylene glycol, phenyl ethyl alcohol, ethanol, isopropyl alcohol, glycerin, 1,3-butanediol, 1,2-propane diol, isoprene glycol, and other polar solvents such as water, alcohols, ketones and mixtures thereof. The conductive material is more preferably selected from propylene glycol, ethanol, and mixtures thereof, and is most preferably propylene glycol.

Branched Polyether-Polydiorganosiloxane Emulsifier

The present emulsion comprises a branched polyether-polydiorganosiloxane emulsifier in an amount sufficient, but not unnecessarily irritating to the skin, for providing a stable polyol-in-silicone emulsion. Preferably, the branched polyether-polydiorganosiloxane emulsifier is comprised in a total amount of from about 0.1% to about 5.0%, more preferably about 0.2% to about 4.0%, of the polyol-in-silicone emulsion.

The branched polyether-polydiorganosiloxane emulsifier of the present invention is a nonionic polysiloxane copolymer having emulsifying ability, comprising a methylpolysiloxane backbone, one or more polydiorganosiloxane branches, and a poly(oxyalkylene)methylpolysiloxane moiety; having an HLB from about 2 to about 8, and a molecular weight of from about 2,000 to about 10,000. The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in “Surfactant Science Series, Vol. 1: Nonionic Surfactants”, pp 606-13, M. J. Schick (Marcel Dekker Inc., New York, 1966).

Suitable branched polyether-polydiorganosiloxane emulsifiers herein are those which have the following formulation (I): 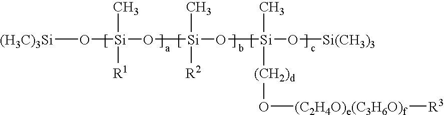
(I)
wherein R¹ is an alkyl group having from about 1 to about 20 carbons; R² is 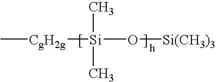
wherein g is from about 1 to about 5, and h is from about 5 to about 20; R³ is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.
Highly preferred commercially available branched polyether-polydiorganosiloxane emulsifiers include PEG-9 polydimethylsiloxyethyl Dimethicone, having an HLB of about 4 and a molecular weight of about 6,000 having a tradename KF 6028 available from ShinEtsu Chemical.
Alkyl Dimethicone Copolyol Emulsifier

The present emulsion comprises an alkyl dimethicone copolyol emulsifier in an amount sufficient, but not unnecessarily irritating to the skin, for providing a stable polyol-in-silicone emulsion. Preferably, the alkyl dimethicone copolyol emulsifier is comprised in a total amount of from about 0.1% to about 3.0%, more preferably about 0.2% to about 2.5%, of the polyol-in-silicone emulsion. Surprisingly, the combination of the aforementioned branched polyether-polydiorganosiloxane emulsifier and alkyl dimethicone copolyol emulsifier provides a polyol-in-silicone emulsion that is stable in the aspects of: smaller emulsion droplet size, stability at different temperature, stability over a longer period of time, lack of separation, and consistent viscosity. In one embodiment, the aforementioned branched polyether-polydiorganosiloxane emulsifier and alkyl dimethicone copolyol emulsifier is included at a weight ratio of from about 1:2 to about 1:0.25.

The alkyl dimethicone copolyol of the present invention is a nonionic polysiloxane copolymer having emulsifying ability, comprising a methylpolysiloxane moiety, an alkyl methylpolysiloxane moiety, and a poly(oxyalkylene)methylpolysiloxane moiety; having an HLB from about 4 to about 6, and a molecular weight of from about 10,000 to about 20,000, wherein the alkyl group is made of from about 10 to about 22 carbons.

Suitable alkyl dimethicone copolyols herein are those which have the following formulation (II): 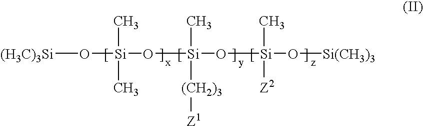
wherein Z¹ is O(C₂H₄O)ₚ(C₃H₆O)_{q}H, p is from 0 to about 50, q is from 0 to about 30, wherein p and q are not 0 at the same time; x is from 1 to about 200, y is from 1 to about 40, and z is from 1 to about 100, and Z² is an alkyl group having from about 10 to about 22 carbons, preferably from about 16 to about 18 carbons.

Highly preferred alkyl dimethicone copolyols include cetyl dimethicone copolyol and stearyl dimethicone copolyol. A highly preferred commercially available alkyl dimethicone copolyol includes cetyl dimethicone copolyol, also called Methylpolysiloxane Cetylmethylpolysiloxane Poly(oxyethylene oxypropylene) Methylpolysiloxane Copolymer, having an HLB of about 5 and a molecular weight of about 13,000 having a tradename ABIL EM90 available from Goldschmidt Personal Care.

Thickeners

The present emulsion comprises a thickener in an amount sufficient, but not unnecessarily irritating to the skin, for providing a stable polyol-in-silicone emulsion. The type and amount of thickeners will vary based on the additional components for comprising in the desired product form. Preferably, the thickener is comprised in a total amount of from about 0.2% to about 20%, more preferably about 0.4% to about 15%, of the polyol-in-silicone emulsion.

Suitable thickeners can be selected from the group consisting of silicones, waxes, clays, silicas, salts, natural and synthetic esters, fatty alcohols, and mixtures thereof. Nonlimiting examples of these thickeners are described below. In one preferred embodiment, the thickener is an organophilically modified clay.

Suitable silicones include alkyl siloxane gellants, silicone elastomers, high molecular weight dimethicones (fluids greater than 1000 mPas), and high molecular weight alkyl, hydroxyl, carboxyl, amino, and/or fluoro-substituted dimethicones (fluids greater than 1000 mPas). Preferred silicone gellants are described in U.S. Pat. Nos. 5,654,362 and 5,880,210, and include cyclomethicone and dimethicone crosspolymers (e.g., Dow Corning 9040). Preferred silicone elastomers include Dimethicone PEG-10/15 crosspolymer (KSG 210 available from ShinEtsu Chemical).

Waxes can be defined as lower-melting organic mixtures or compounds of high molecular weight, solid at room temperature and generally similar in composition to fats and oils except that they contain no glycerides. Some are hydrocarbons, others are esters of fatty acids and alcohols. Suitable waxes may be selected from the group consisting of natural waxes including animal waxes, vegetable waxes, and mineral waxes, and synthetic waxes including petroleum waxes, ethylenic polymers, hydrocarbon waxes (e.g., Fischer-Tropsch waxes), ester waxes, silicone waxes, and mixtures thereof. Synthetic waxes include those disclosed in Warth, Chemistry and Technology of Waxes, Part 2, Reinhold Publishing (1956); herein incorporated by reference.

Specific examples of waxes include beeswax, lanolin wax, shellac wax, carnauba, candelilla, bayberry, jojoba esters, behenic acid waxes (e.g., glyceryl behenate which is available from Gattifosse as Compritol®), ozokerite, ceresin, paraffin, microcrystalline waxes, polyethylene homopolymers, polymers comprising ethylene oxide or ethylene (e.g., long chained polymers of ethylene oxide combined with a dihydric alcohol, namely polyoxyethylene glycol, such as Carbowax available from Carbide and Carbon Chemicals company; long-chained polymers of ethylene with OH or another stop length grouping at end of chain, including Fischer-Tropsch waxes as disclosed in Warth, supra, at pages 465-469 and specifically including Rosswax available from Ross Company and PT-0602 available from Astor Wax Company), C₂₄₋₄₅ alkyl methicones, C₈ to C₅₀ hydrocarbon waxes, alkylated polyvinyl pyrrolidones (e.g., “Ganex” alkylated polyvinylpyrrolidines available from the ISP Company), fatty alcohols from C20 to C60 (e.g., “Unilins”, available from Petrolite Corporation), and mixtures thereof.

Water dispersible and oil dispersible clays may be useful to provide thickening. Suitable clays can be selected, e.g., from montmorillonites, bentonites, hectorites, attapulgites, sepiolites, laponites, silicates and mixtures thereof.

Suitable water dispersible clays include bentonite and hectorite (such as Bentone EW, LT from Rheox); magnesium aluminum silicate (such as Veegum from Vanderbilt Co.); attapulgite (such as Attasorb or Pharamasorb from Engelhard, Inc.); laponite and montmorillonite (such as Gelwhite from ECC America); and mixtures thereof.

Suitable oil dispersible clays include organophilically modified bentonites, hectorites and attapulgites. Specific commercially available examples of these clays include Bentone 34 (Rheox Corp.)—Quaternium-18 Bentonite; Tixogel VP (United Catalysts)—Quaternium-18 Bentonite; Bentone 38; Bentone 38V (Rheox Corp.)—Quaternium-18 Hectorite; Bentone SD-3 (Rheox Corp.)—Dihydrogenated Tallow Benzylmonium Hectorite; Bentone 27; Bentone 27V (Rheox Corp.)—Stearalkonium Hectorite; Tixogel LG (United Catalysts)—Stearalkonium Bentonite; Claytone 34 (Southern Clay) Quaternium-18 Bentonite; Claytone 40 (Southern Clay) Quaternium-18 Bentonite; Claytone AF (Southern Clay) Stearalkonium Bentonite; Claytone APA (Southern Clay) Stearalkonium Bentonite; Claytone GR (Southern Clay) Quaternium-18/Benzalkonium Bentonite; Claytone HT (Southern Clay) Quaternium-18/Benzalkonium Bentonite; Claytone PS (Southern Clay) Quaternium-18/Benzalkonium Bentonite; Claytone XL (Southern Clay) Quaternium-18 Bentonite; and Vistrol 1265 (Cimbar)—Organophilic Attapulgite. These organophilic clays can be purchased as pre-dispersed organophilic clay in either an oil or an organic solvent. The materials are in the form of a heavy paste that can be readily dispersed into the formulation. Such materials include Mastergels by Rheox, United Catalysts, and Southern Clay.

Other thickeners include fumed silicas and alkali metal or ammonium halides. Examples of fumed silicas include Aerosil 200, Aerosil 300, and the alkyl-substituted fumed silicas such as Aerosil R-100, 200, 800, and 900 series of materials, all available from the DeGussa Corporation.

Other thickeners useful herein include modified dextrin such as stearoyl inulin (Rheopearl ISK available from Chiba Flour Milling).

Product Forms and Particular Compositions

The polyol-in-silicone emulsion of the present invention may be formulated into a variety of product forms useful for application on the skin. These product forms include color cosmetic compositions, skin care compositions, UV protection and self-tanning products. Color cosmetic compositions herein include foundations, blushers, hilighters, eyeshadows, and make-up base. Skin care compositions herein include skin lotions, milk lotions, spray lotions, creams and gels.

In one aspect, the present invention relates to a composition comprising the aforementioned polyol-in-silicone emulsion and further from about 0.1% to about 35% of a powder for providing color or skin feel benefit. In another aspect, the present invention relates to a composition comprising the aforementioned polyol-in-silicone emulsion and further from about 0.5% to about 20% of a film forming agent for wear resistance. In yet another aspect, the present invention relates to a composition comprising the aforementioned polyol-in-silicone emulsion and further from about 0.001% to about 20% of a skin active agent.

Powder

The compositions of the present invention may comprise a powder, which is generally defined as dry, particulate matter having a particle size of from 0.001 to 150 microns, preferably 0.01 to 100 microns. The powder materials may be colored or non-colored (e.g., white or essentially clear), and may provide one or more benefits to the composition or skin such as coloration, light diffraction, oil absorption, translucency, opacification, pearlescence, matte appearance, lubricious feel, skin coverage and the like. These materials are well known in the art and are commercially available. Selection of the particular type and level of a given powder material for a particular purpose in a given product is within the skill of the artisan. Such materials are typically used in an amount of from about 0.2% to about 35% preferably from about 0.5% to about 30% by weight, more preferably from about 1% to about 25% by weight of the composition.

Other useful powder materials include talc, mica, titanated mica (mica coated with titanium dioxide), iron oxide titanated mica, magnesium carbonate, calcium carbonate, magnesium silicate, silica (including spherical silica, hydrated silica and silica beads), titanium dioxide, zinc oxide, nylon powder, polyethylene powder, ethylene acrylates copolymer powder, methacrylate powder, polystyrene powder, silk powder, crystalline cellulose, starch, bismuth oxychloride, guanine, kaolin, chalk, diatomaceous earth, microsponges, boron nitride and the like. Additional powders useful herein are described in U.S. Pat. No. 5,505,937 issued to Castrogiovanni et al. Apr. 9, 1996.

Of the components useful as a matte finishing agents, low luster pigment, talc, polyethylene, hydrated silica, kaolin, titanium dioxide, titanated mica and mixtures thereof are preferred. Also useful are titanium dioxide and zinc oxide having particle sizes of sunscreen grade.

Micas, boron nitride and ethylene acrylates copolymer (e.g., EA-209 from Kobo) are preferred for imparting optical blurring effects through light diffraction and improving skin feel, e.g., by providing a lubricious feel. Another particulate material for improving skin feel is SPCAT 12 (a mixture of talc, polyvinylidene copolymer, and isopropyl titanium triisostearate).

Preferred powders for absorbing oil are spherical, nonporous particles, more preferably having a particle size less than 25 microns. Examples of some preferred oil absorbing powders are Coslin C-100 (a spherical oil absorber commercially available from Englehard), Tospearl 145A (polymethylsilsesquioxane available from GE Toshiba Silicones), Powder La Vie (sericite deposited by hudroxyapatite and zinc oxide, commercially available from Miyoshi Kasei), ethylene acrylates copolymer such as noted above, and SPCAT 12.

The powders may be surface treated with one or more agents, e.g., with lecithin, amino acids, mineral oil, silicone oil, or various other agents, which coat the powder surface, for example, to render the particles hydrophobic or hydrophilic. Such treatment may be preferred to improve ease of formulation and stability.

Film Forming Polymers

The compositions of the present invention may comprise a film forming polymer, for imparting wear and/or transfer resistant properties. When included, such materials are typically used in an amount of from about 0.5% to about 20% preferably from about 0.5% to about 10% by weight, more preferably from about 1% to about 8% by weight of the composition. Preferred polymers form a non-tacky film which is removable with water used with cleansers such as soap.

Examples of suitable film forming polymeric materials include:
a) sulfopolyester resins, such as AQ sulfopolyester resins, such as AQ29D, AQ35S, AQ38D, AQ38S, AQ48S, and AQ55S (available from Eastman Chemicals);
b) polyvinylacetate/polyvinyl alcohol polymers, such as Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) polyvinylpyrrolidones (PVP), including Luviskol K17, K30 and K90 (available from BASF), water soluble copolymers of PVP, including PVP/VA S-630 and W-735 and PVP/dimethylaminoethylmethacrylate Copolymers such as Copolymer 845 and Copolymer 937 available from ISP, as well as other PVP polymers disclosed by E. S. Barabas in the Encyclopedia of Polymer Science and Engineering, 2 Ed. Vol. 17 pp. 198-257;
e) high molecular weight silicones such as dimethicone and organic-substituted dimethicones, especially those with viscosities of greater than about 50,000 mPas;
   f) high molecular weight hydrocarbon polymers with viscosities of greater than about 50,000 mPas;
g) organosiloxanes, including organosiloxane resins, fluid diorganopolysiloxane polymers and silicone ester waxes.

Examples of these polymers and cosmetic compositions containing them are found in PCT publication Nos. WO96/33689, published Oct. 31, 1996; WO97/17058, published May 15, 1997; and U.S. Pat. No. 5,505,937 issued to Castrogiovanni et al. Apr. 9, 1996, all incorporated herein by reference. Additional film forming polymers suitable for use herein include the water-insoluble polymer materials in aqueous emulsion and water soluble film forming polymers described in PCT publication No. WO98/18431, published May 7, 1998, incorporated herein by reference. Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons.

Preferred film forming polymers include organosiloxane resins comprising combinations of R₃SiO_{1/2} “M” units, R₂SiO “D” units, RSiO_{3/2} “T” units, SiO₂ “Q” units in ratios to each other that satisfy the relationship RₙSiO_{(4-n)/2} where n is a value between 1.0 and 1.50 and R is a methyl group. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrofunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as Wacker 803 and 804 available from Wacker Silicones Corporation of Adrian Mich., KP545 from Shin-Etsu Chemical and G. E. 1170-002 from the General Electric Company.

Other materials for enhancing wear or transfer resistance include trimethylated silica. Suitable silicas of this type and cosmetic compositions containing them are described in U.S. Pat. No. 5,800,816 issued to Brieva et al., incorporated herein by reference.

Skin Active Agents

The compositions of the present invention may comprise a safe and effective amount of a skin active agent. The term “skin active agent” as used herein, means an active ingredient which provides a cosmetic and/or therapeutic effect to the area of application on the skin, hair, or nails. The skin active agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, sunscreening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present composition comprises from about 0.001% to about 30%, preferably from about 0.001% to about 10% of at least one skin active agent.

The type and amount of skin active agents are selected so that the inclusion of a specific agent does not affect the stability of the composition. For example, hydrophilic agents may be incorporated in an amount soluble in the polyol phase, while lipophilic agents may be incorporated in an amount soluble in the silicone phase.

Skin lightening agents useful herein refer to active ingredients that improve hyperpigmentation as compared to pre-treatment. Useful skin lightening agents herein include ascorbic acid compounds, vitamin B₃ compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C available from Roche.

Vitamin B₃ compounds useful herein include, for example, those having the formula: 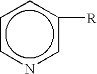
wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.

Other skin active agents useful herein include those selected from the group consisting of panthenol, tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

UV Absorbing Agents

The compositions of the present invention may comprise a safe and effective amount of a UV absorbing agent. A wide variety of conventional UV protecting agent are suitable for use herein, such as those decribed in U.S. Pat. No. 5,087,445, Haffey et al, issued Feb. 11, 1992; U.S. Pat. No. 5,073,372, Turner et al, issued Dec. 17, 1991; U.S. Pat. No. 5,073,371, Turner et al., issued Dec. 17, 1991; and Segarin, et al, at Chapter VIII, pages 189 et seq., of Cosmetics Science and Technology (1972). When included, the present composition comprises from about 0.5% to about 20%, preferably from about 1% to about 15% of a UV absorbing agent.

UV absorbing agents useful herein are, for example, 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof.

Additional Components

The compositions hereof may further contain additional components such as are conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits).

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

Examples of suitable topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

Electrostatically Sprayable

Electrostatic spraying of color cosmetics and skin care compositions has been proposed as a means for more efficient consumption and product activity, control over application, ease and cleanliness/hygiene of application, and improved finish. Electrostatic spraying involves raising the composition to be sprayed to a high electric potential in a spray nozzle to cause the composition to atomize as a spray of electrically charged droplets. The electrically charged droplets seek the closest earthed object to discharge their electric charge, which can be arranged to be the desired spray target.

In one preferred embodiment, the polyol-in-silicone emulsion of the present invention and compositions comprising thereof are electrostatically sprayable. In order to be electrostatically sprayable, a composition must have a resistivity which enables atomization as a sprayed. Stability of compositions comprising emulsions and additional powders are particularly challenging, as such compositions tend to separate, and the powder may settle out. The resultant non-homogeneous fluid exhibits poor spray quality when sprayed electrostatically. The present compositions also provide improved spray quality under conditions of electrostatic spraying.

The compositions hereof are suitably directly applied to the skin by electrostatic spray techniques. In general, this method involves raising the composition to be sprayed to a high electric potential in a spray nozzle to cause the composition to atomize as a spray of electrically charged droplets. The electrically charged droplets seek the closest earthed object to discharge their electric charge, which can be arranged to be the desired spray target.

Compositions to be delivered using the present invention are preferably generally liquid in form. Any adjunct materials which are present may be liquid, solid or semi-solid at room temperature, though they should be selected so as to permit electrostatic spraying of the composition. For enhancing electrostatic spraying, preferred compositions have a solids content of about 35 weight % or less. In this regard, “solids” refers to particulate materials which are not soluble or miscible in the composition, and includes particulate pigments and oil absorbers.

In one highly preferred embodiment, the present invention relates to an electrostatically sprayable composition suitable for use as a color cosmetic composition such as foundation, blusher, or highlight, comprising the following components:
a) from about 2% to about 90% of the continuous silicone phase;
b) from about 2% to about 90% of the discontinuous polyol phase;
c) from about 0.1% to about 5% of the branched polyether-polydiorganosiloxane emulsifier;
d) from about 0.1% to about 3% of the alkyl dimethicone copolymer emulsifier;
e) from about 0.2% to about 20% of the thickener for stabilizing the composition;
f) from about 0.2% to about 35% of the powder; and
g) from about 0.5% to about 20% of the film forming polymer.

For use in the present invention, the hardware and electrical componentry and circuitry may be of any suitable construction and design. Preferred devices include an apparatus suitable for small-scale personal use which has a reservoir for containing the present composition, at least one delivery means, e.g., a nozzle, in communication with the reservoir; a high voltage generator generating voltage in the range of 1 to 26 kilovolts (e.g., from 12 to 26 kilovolts) powered from a portable or non-portable (preferably portable) electricity source; and control means for selectively applying the high voltage from the generator to the at least one delivery means. In spray of the charged droplets, preferably of from about 0.01 to about 5000 Mega-ohm-cm, more preferably from about 0.01 to about 2000 Mega-ohm-cm, most preferably from about 0.1 to about 500 Mega-ohm-cm. Resistivity is measured using standard, conventional apparatus and methods, generally at 25 degree C. Resistivity can be adjusted as necessary by varying the relative levels of insulating materials and conductive materials. The compositions must also have a viscosity which permits electrostatically spraying, namely sufficiently high to minimize wicking of the composition droplets as they are applied, yet fluid enough to be atomized when charged. Preferably the viscosity is in the range of from about 0.1 to about 50,000 mPas, more preferably from about 0.5 to about 20,000 mPas, most preferably from about 5 to about 10,000 mPas (at 25 degree C., using 60 mm parallel plate with 0.5 mm gap at rate of 10 sec⁻¹).

In summary, for a composition useful for skin application to be electrostatically sprayable, the composition typically comprises: a liquid insulating material, a conductive material, and a thickener for stabilizing and adjusting the viscosity of the composition. By “insulating” it is meant that a material would not itself be suitable for electrostatic spraying (that is, it would not be able to cause sufficient alignment of the dipole molecules in the field to result in the subsequent, necessary net force), typically having a resistivity of greater than about 2000 Mega-ohm-cm, more typically greater than about 5000 Mega-ohm-cm. Preferred insulating materials have a viscosity of about 10,000 mPas or less. The conductive material ensures that the composition as a whole can, when in the presence of a non-uniform electric field, generate dielectrophoretic forces great enough to pull the composition toward the region of highest field intensity (hence creating an electrostatic spray). The conductive material preferably has a resistivity of less than 5000 Mega-ohm-cm, more preferably less than about 2000 Mega-ohm-cm, most preferably less than about 500 Mega-ohm-cm. This material preferably also has a relaxation time which is sufficiently long to enable a spray wherein all of the droplets have a particle size of less than 300 microns according to standard light microscopy techniques. The conductive material preferably has a relaxation time of from about 1E-7 to 1 seconds, more preferably from about 1E-6 to 1E-2 seconds, most preferably from about 1E-5 to 1E-3 seconds.

The present polyol-in-silicone emulsion is electrostatically sprayable when the continuous silicone phase is capable of acting as a suitable insulating material, the discontinuous polyol phase is capable of acting as a suitable conductive material, and the thickener provides a suitable viscosity. It has been surprisingly found that, when meeting such conditions, the emulsion of the present invention provides improved stability even when electrostatically sprayed. Further, it has been found that compositions comprising the present emulsion and additional components, such as powders, also provide improved stability when electrostatically use, the control means is actuated to electrostatically spray the topical composition from the at least one delivery means directly onto the skin at an intended site.

As will be appreciated by persons skilled in the art, particular constructional features and design and electrical and other operating parameters of such apparatuses may be selected or adjusted as necessary, in accordance with the desired functioning characteristics, as for example dictated by the composition to be sprayed and/or the needs or wishes of a user. Features of the apparatus which may be so selected and/or adjusted include for example: voltage generated by the high voltage generator and power source, electric field strength in or in the region of the product delivery means, flow rate of the product to be sprayed from the reservoir to and out of the delivery means, size and configuration of the delivery means itself and construction and properties of any product feed mechanism utilized between the reservoir and the output of the delivery means.

The size and configuration of the one or more delivery means in the apparatus may be of any suitable form and again may be selected in association with other parameters to give an optimized functioning electrostatic spray delivery system. Commonly the or each delivery means will be in the form of a nozzle, preferably of insulating or semi-insulating material such as plastics or various polymers, as is well known in the art. In one preferred form of nozzle, a conduit for carrying the composition to be sprayed terminates in an orifice at the tip of the nozzle, from which orifice the composition is ejected for example initially as a ligament but in any event eventually dispersing as a spray of charged droplets. The orifice preferably has a diameter of not greater than about 800 microns (e.g., from 508-762 microns). Even more preferably the orifice has a diameter of from about 500 to about 750 microns.

The delivery means may advantageously include metering means to provide a dosing mechanism for delivering a predetermined fixed amount of composition from the or each nozzle. Such an expedient may for example be useful in conjunction with a system having a controlled flow rate. Preferably the or each delivery means is in communication, i.e. preferably fluid communication, with the reservoir by virtue of composition feed means. In one preferred form, such feed means may comprise an insulator having a channel between the nozzle and the reservoir, through which the composition to be sprayed flows before reaching the point of high electric field strength where it is dispersed as a charged spray of droplets or particles. In another preferred form the feed means may comprise a hollow conduit through which the composition passes under the effect of capillary action.

The apparatus preferably includes a trigger (i.e. a manual control means) or alternatively an automatic control means to selectively apply the high voltage from the generator to the or each delivery means to electrostatically spray the composition onto the skin. Any other suitable control means however, e.g. which automatically control actuation of the system, may be used, as will be appreciated by persons skilled in the art.

The deposition of the composition on the skin, including spray droplet size and spacing and skin coverage, is influenced by the product spray flow rate, the rate of product application to the skin, and the amount of product applied to the skin. In general, droplet size increases with increasing resistivity, decreasing voltage, and increasing flow rate, spacing increases with increasing voltage and decreasing deposition amount, and coverage increases with increasing flow rate and increasing deposition amount.

Optimum flow rates of composition to be sprayed will generally depend upon the composition itself, and may be selected appropriately on that basis preferably so as to avoid sensory negatives. Also, as already mentioned with respect to viscosity of the sprayable material, a suitable flow rate may be selected depending upon the particular delivery regime and/or habit or needs of a user. Generally it will be desired to utilize lower flow rates with concentrated materials in order to better control the deposition of the composition. In general, as the flow rate increases it will be desired to utilize a higher voltage in order to provide optimal sprayability and small sprayed droplet sizes. In a preferred embodiment, the present composition is sprayed at a flow rate of from about 0.1 to about 100 ml/hr, preferably from about 1 to about 30 ml/hr, a voltage of from about 1 kV to about 20 kV, preferably from about 3 kV to about 20 kV, and an application rate of from about 0.01 mg composition/cm² of skin to about 12 mg composition/cm² of skin. Relatively high solids compositions such as foundations are typically applied at a rate of about 1 mg/cm² skin; relatively low solids compositions such as skin lotions are typically applied at a rate of about 5 to 6 mg/cm² skin. Relatively low solids compositions such as skin lotions are typically delivered at a flow rate of from about 50 to about 60 ml/hr. Relatively high solids compositions such as foundations are preferably electrostatically sprayed at said application rate, at a flow rate of from about 1 to about 30 ml/hr and a voltage of from about 6 kV to about 20 kV. An exemplary application amount of a highly preferred foundation embodiment of the present composition is about 0.8 mg/cm², which tends to provide about 30-40% skin coverage.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

Compositions

| Ingredient | Ex 1 | Ex 2 | Ex 3 | Ex 4 | Ex 5 |
|---|---|---|---|---|---|
| Group A | | | | | |
| Cyclomethicone 245 | 26.80 | 26.80 | 25.30 | 27.30 | 23.80 |
| PEG-9 Polydimethylsiloxyethyl | 2.00 | 2.00 | 1.50 | 1.50 | 2.00 |
| Dimethicone¹ | | | | | |
| Cetyl Dimethicone Copolyol² | 1.00 | 0.60 | 1.50 | 1.00 | 1.00 |
| Dimethicone PEG- | | | | | 1.00 |
| 10/15 Crosspolymer³ | | | | | |
| Group B | | | | | |
| Hectorite Clay⁴ | 1.00 | 1.00 | 1.50 | 1.00 | 0.80 |
| Propylene Carbonate | 0.25 | 0.25 | 0.25 | 0.25 | 0.25 |
| Stearoyl Inulin⁵ | | | | 0.50 | 0.25 |
| Cyclomethicone 245 | | | | 2.50 | 1.25 |
| Group C | | | | | |
| Treated Iron Oxides | 1.46 | 1.46 | 1.46 | 1.46 | 1.46 |
| Boron Nitride⁶ | 1.50 | 1.50 | 1.50 | 1.50 | 1.50 |
| Talc - Dimethicone Treated | 1.20 | 0.70 | 0.70 | 1.20 | 1.20 |
| Organosiloxane resin⁷ | 2.50 | | 2.50 | 2.50 | |
| Acrylates/Dimethicone Copolymer⁸ | | 2.50 | | | 2.50 |
| Titanium Dioxide - Dimethicone | 4.50 | 4.50 | 4.00 | 4.50 | 4.00 |
| Treated | | | | | |
| Polymethylsilsesquioxane⁹ | 1.50 | 1.50 | 1.50 | 1.50 | 1.50 |
| Silica - Dimethicone treated | | 2.00 | 2.00 | 1.00 | |
| Mica and Hydroxyapatite and Zinc | | | 1.50 | 2.50 | |
| oxide¹⁰ | | | | | |
| Micronized Titanium Oxide - | | | 0.50 | | 1.0 |
| Silicone coated | | | | | |
| Micronized Zinc Oxide - | | | | | 0.5 |
| Dimethicone coated | | | | | |
| Niacinamide | | | 1.00 | | 1.50 |
| Group D | | | | | |
| Ethanol | | 5.00 | | 3.0 | 5.00 |
| Deionized Water | | 4.00 | | 3.0 | 5.00 |
| Propylene Glycol | 55.79 | 45.69 | 52.29 | 43.29 | 43.99 |
| Colorants, perfume, preservatives | 0.5 | 0.5 | 1 | 0.5 | 0.5 |
| Ingredient | Ex 6 | Ex 7 | Ex 8 | Ex 9 | Ex 10 |
| Group A | | | | | |
| Cyclomethicone 245 | 26.80 | 26.05 | 25.30 | 26.30 | 23.80 |
| PEG-9 Polydimethylsiloxyethyl | 2.00 | 2.00 | 1.50 | 1.50 | 2.00 |
| Dimethicone¹ | | | | | |
| Cetyl Dimethicone Copolyol² | 1.00 | 0.60 | 1.50 | 1.00 | 1.00 |
| Octyl methoxy cinnamate | 8.00 | | 4.00 | 8.00 | |
| Avobenzone¹¹ | 1.00 | | 0.75 | 1.00 | |
| Dimethicone PEG- | | | | 0.50 | |
| 10/15 Crosspolymer³ | | | | | |
| Group B | | | | | |
| Hectorite Clay⁴ | 1.00 | 1.00 | 1.50 | 0.5 | 0.80 |
| Propylene Carbonate | 0.25 | 0.25 | 0.25 | 0.12 | 0.25 |
| Stearoyl Inulin⁵ | | 0.15 | | 0.25 | 0.50 |
| Cyclomethicone 245 | | 0.75 | | 1.25 | 2.50 |
| Group C | | | | | |
| Treated Iron Oxides | 1.46 | 1.46 | 1.46 | 1.46 | 1.46 |
| Boron Nitride⁶ | 1.50 | 2.00 | | 0.50 | 1.50 |
| Talc - Dimethicone Treated | 1.20 | 0.70 | 0.70 | 0.30 | 0.70 |
| Organosiloxane resin⁷ | 2.50 | | 2.50 | 3.50 | |
| Acrylates/Dimethicone Copolymer⁸ | | 2.50 | | | 2.50 |
| Titanium Dioxide - Dimethicone | 4.50 | 3.00 | 4.00 | 1.50 | 5.00 |
| Treated | | | | | |
| Polymethylsilsesquioxane⁹ | 1.50 | 1.50 | 2.00 | 2.50 | 1.50 |
| Silica - Dimethicone treated | | 0.50 | 1.00 | 0.25 | |
| Mica and Hydroxyapatite and | | 1.00 | 1.00 | 0.50 | |
| Zinc oxide¹⁰ | | | | | |
| Micronized Titanium Oxide - | | 5.00 | 3.00 | | 5.00 |
| Silicone coated | | | | | |
| Micronized Zinc Oxide - | | 2.00 | 1.50 | | 2.00 |
| Dimethicone coated | | | | | |
| Niacinamide | | | 1.00 | 2.00 | 1.00 |
| Group D | | | | | |
| Ethanol | | 3.00 | | 3.0 | 5.00 |
| Deionized Water | | 3.00 | | 2.0 | 3.00 |
| Propylene Glycol | 46.79 | 43.04 | 46.54 | 41.57 | 39.99 |
| Colorants, perfume, preservatives | 0.5 | 0.5 | 0.5 | 0.5 | 0.5 |

¹PEG-9 polydimethylsiloxyethyl dimethicone: KF6028 from Shin-Etsu Chemical.
²Cetyl Dimethicone Copolyol: Abil EM 90 from Goldschmidt.
³Dimethicone PEG-10/15 Crosspolymer: KSG210 available from Shin-Etsu Chemical.
⁴Hectorite Clay: Bentone 38V available from Elementis Specialties.
⁵Stearoyl Inulin: Rheopearl ISK available from Chiba Flour Milling.
⁶Boron Nitride: Torayceram T-BN-C available form Toray Industries.
⁷Organisloxane resin: MQ Resin (0.7:1 ratio M:Q) available as SR 1000 from General Electric.
⁸Acrylates/Dimethicone Copolymer: KP545 available from Shin-Etsu Chemical.
⁹Polymethylsilsesquioxane: Tospearl 145A available from GE-Toshiba Silicones.
¹⁰Mica and Hydroxyapatite and Zinc oxide: Powder La Vie available from Miyoshi Kasei.
¹¹Avobenzone: Parsol 1789 available from Givaudan.

Preparation of Composition

The compositions above may be made by any suitable method known to the artisan. The compositions may be made as follows: Combine the Group A ingredients and mix well with a homogenizer. Prepare Group B ingredients by dispersing hectorite clay and activator at medium shear or dissolving Rheopearl ISK in silicone at about 65 degree C. Add Group B ingredients to Group A and mix them for about 10 minutes. Add Group C ingredients slowly to Group A and B while mixing at medium speed and then high speed after addition is completed. Assist with additional hand mixing if necessary. Add premixed Group D to Group A, B and C for emulsifying in about 10 minutes. Mix for additional about 5 minutes after addition is completed. Allow batch to reach ambient conditions and pour into appropriate container.

Use of Compositions

Examples 1-10 above provide polyol-in-silicone emulsion type compositions that are stable in the aspects of: smaller emulsion droplet size, stability at different temperature, stability over a longer period of time, lack of separation, and consistent viscosity. Further, Examples 1-10 are electrostatically sprayable.

It is understood that the foregoing detailed description of examples and embodiments of the present invention are given merely by way of illustration, and that numerous modifications and variations may become apparent to those skilled in the art without departing from the spirit and scope of the invention; and such apparent modifications and variations are to be included in the scope of the appended claims.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A polyol-in-silicone emulsion comprising:
a) a continuous silicone phase;
b) a discontinuous polyol phase;
c) a branched polyether-polydiorganosiloxane emulsifier;
d) an alkyl dimethicone copolymer emulsifier; and
e) a thickener for stabilizing the composition.

2. The emulsion of claim 1 wherein the continuous silicone phase comprises a cyclic polyalkylsiloxane having the formula [SiR₂—O]ₙ wherein R is methyl and n is an integer of from about 4 to about 6.

3. The emulsion of claim 1 wherein the discontinuous polyol phase is selected from the group consisting of propylene glycol, butylene glycol, dipropylene glycol, phenyl ethyl alcohol, ethanol, isopropyl alcohol, glycerin, 1,3-butanediol, 1,2-propane diol, isoprene glycol, water, acetone, and mixtures thereof.

4. The emulsion of claim 3 wherein the discontinuous polyol phase comprises propylene glycol.

5. The emulsion of claim 1 wherein the branched polyether-polydiorganosiloxane emulsifier is selected from general formulae (I) 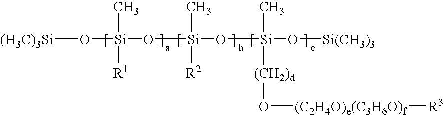
(I)
wherein R¹ is an alkyl group having from about 1 to about 20 carbons; R² is 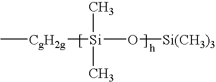
wherein g is from about 1 to about 5, and h is from about 5 to about 20; R³ is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.

6. The emulsion of claim 1 wherein the alkyl dimethicone copolymer emulsifier is selected from general formulae (II) 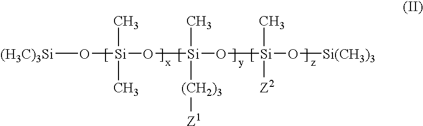
wherein Z¹ is O(C₂H₄O)ₚ(C₃H₆O)_{q}H, p is from 0 to about 50, q is from 0 to about 30, wherein p and q are not 0 at the same time; x is from 1 to about 200, y is from 1 to about 40, and z is from 1 to about 100, and Z² is an alkyl group having from about 10 to about 22 carbons.

7. The emulsion of claim 1 wherein the thickener is selected from the group consisting of silicones, waxes, clays, silicas, salts, natural and synthetic esters, fatty alcohols, and mixtures thereof.

8. The emulsion of claim 7 wherein the thickener is an organophilically modified clay.

9. A composition comprising the emulsion of claim 1 and a powder.

10. A composition comprising the emulsion of claim 1 and a film forming polymer.

11. The composition of claim 10 wherein the film forming polymer is MQ resin.

12. A composition comprising the emulsion of claim 1 and a skin active agent.

13. A composition comprising the emulsion of claim 1 and a UV absorbing agent.

14. A composition comprising the emulsion of claim 1, the entire composition comprising:
a) from about 0.2% to about 90% of the continuous silicone phase;
b) from about 0.2% to about 90% of the discontinuous polyol phase;
c) from about 0.1% to about 5% of the branched polyether-polydiorganosiloxane emulsifier;
d) from about 0.1% to about 3% of the alkyl dimethicone copolymer emulsifier;
e) from about 0.2% to about 20% of the thickener for stabilizing the composition;
f) from about 0.2% to about 35% of the powder; and
g) from about 0.5% to about 20% of the film forming polymer.

15. The emulsion of claim 1, wherein the continuous silicone phase is a liquid insulating material; the discontinuous polyol phase is a conductive material; and wherein the emulsion is electrostatically sprayable.

16. The electrostatically sprayable emulsion of claim 15 wherein the emulsion can be electrostatically sprayed at a flow rate of from about 0.1 to about 100 ml/hr, a voltage of from about 1 kV to about 20 kV, and an application rate of from about 0.01 mg emulsion/cm2 skin to about 12 mg emulsion/cm2 skin.

17. The composition of any of claims 9 through 14, wherein the continuous silicone phase is a liquid insulating material; the discontinuous polyol phase is a conductive material; and wherein the composition is electrostatically sprayable.

18. The electrostatically sprayable composition of claim 17 wherein the composition can be electrostatically sprayed at a flow rate of from about 0.1 to about 100 ml/hr, a voltage of from about 1 kV to about 20 kV, and an application rate of from about 0.01 mg composition/cm² skin to about 12 mg composition/cm² skin.
